(19) United States
(12) Patent Application Publication
(10) Pub. No.: US 2007/0219425 A1
(43) Pub. Date: Sep. 20, 2007

(54) Waste Disposal Device

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/465,063

(22) Filed: Aug. 16, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.

(51) Int. Cl. A61B 5/00

(52) U.S. Cl. 600/300

(57) ABSTRACT

A waste storage device that includes a storage compartment, a toilet seat with an opening providing access to the storage compartment, and a sealing mechanism operative to isolate deposited waste events within individually sealed portions of the storage compartment. The storage compartment preferably retains a plurality of the individually sealed portions in a connected series along a length of the storage compartment. The waste storage device is useful in a process that accepts consideration-based private civil security subscriptions from subscribers with respect to providing civilly-catastrophic event-based access to a shelter sanitation storage container, which may be the waste disposal device. The process also maintains the shelter sanitation storage container pending a need to permit continued subscription-based access to the shelter sanitation storage container.

### RELATED APPLICATIONS

This comprises a continuation-in-part of each of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application ser. No. 11/381,257;

SUBSCRIPTION-BASED MULTI-PERSON EMERGENCY SHELTER METHOD as filed on May 2, 2006 and having application Ser. No. 11/381,265; and

SUBSCRIPTION-BASED CATASTROPHE-TRIGGERED RESCUE SERVICES FACILITATION METHOD AND APPARATUS as filed on May 2, 2006 and having application No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043; and

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605; and

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

PRIVATELY PROVISIONED SURVIVAL SUPPLIES DELIVERY METHOD as filed on Aug. 15, 2006 and having application Ser. No. 11/464;751;

PRIVATELY PROVISIONED SURVIVAL SUPPLIES SUB UNIT-BASED DELIVERY METHOD as filed on Aug. 15, 2006 and having application Ser. No. 11/464,764;

PRIVATELY PROVISIONED SURVIVAL SUPPLIES ACQUISITION METHOD as filed on Aug. 15, 2006 and having application Ser. No. 11/464,775;

PRIVATELY PROVISIONED SURVIVAL SUPPLIES CONTENT ACQUISITION METHOD as filed on Aug. 15, 2006 and having application Ser. No. 11/464,788;

METHOD TO PRIVATELY PROVISION SURVIVAL SUPPLIES THAT INCLUDE THIRD PARTY ITEMS as filed on Aug. 15, 2006 and having application Ser. No. 11/464,799;

the contents of each of which are fully incorporated herein by this reference.

### TECHNICAL FIELD

The invention relates generally to a waste disposal device and to a method of providing waste disposal in survival-related services.

### BACKGROUND

Modern citizens are today perhaps more at risk of experiencing a serious disruption in their ability to prosper or even to survive en mass than is generally perceived. Providing the necessities of life in general requires a lot of things to operate, more or less, correctly. To put it another way, a serious disruption to any significant element of civilized infrastructure can produce catastrophic results for a broad swath of a given civil entity. Any number of natural and/or non-natural-caused events can greatly disrupt society's infrastructure and corresponding ability to provide one or more life-sustaining resources such as water, nutrition, shelter, and the like.

Many people believe and trust that their government (local, regional, and/or national) will provide for them in the event of such a civilly-catastrophic event. And, indeed, in the long view such is clearly a legitimate responsibility owed by any government to its citizens. That such is a consummation devoutly to be wished, however, does not necessarily make it so. To a large extent one may reasonably argue that governments have forsaken their responsibility to design, fund, implement, or even discuss an effective civil defense program capable of protecting large segments of their populations. In the event of such a civilly-catastrophic event, governments may not be able to ensure that basic sanitary infrastructure, such as water supplies and sewer systems, will be functioning as society normally expects.

Such insights, of course, are not particularly new. Civil preparedness shortcomings occasionally attract public attention and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum.

On the one hand, modern governments typically do little to proactively ensure the bulk survival (let alone the comfort) of their citizens in the face of most civilly-catastrophic events. On the other hand, attempting to take responsible actions to reasonably ensure one's own safety and security can become, in and of itself, nearly a full-time avocation and leave little time to actually enjoy the conveniences and opportunities of modern life. Such individual actions may even be frowned upon by the greater part of society which has grown accustomed and falsely secure with existing efficient just-in-time delivery systems that provide the illusion of plenty while undercutting the perception of risk.

With potential disruptions to sanitary sewer systems comprising a potential consequence of a civilly-catastrophic event, one may look to a variety of portable toilets or other waste disposal systems that are currently on the market as an alternative. One example is the portable, self-contained outhouse that is typically manufactured of plastic and commonly used as a temporary toilet at construction sites or large social gatherings. These portable toilets may be referred to or sold under such brands as Port-ajohn, Port-o-let, Sani-privy, Port-a-san, Porta-potty, or Portaloo, to note but a few. While effective on the job site or a large Fourth-of-July gathering in the park, these portable toilets require liquid disinfectanting chemicals, which render these systems difficult for most individuals to maintain. Likewise, airplanes, buses, campers, and recreational vehicles may also utilize similar devices to retain and hold waste. These devices typically also require chemicals, storage tanks, and proper disposal of the waste, which may be beyond the skills, capabilities, or desires of many individuals, especially in a survival-related situation.

### BRIEF DESCRIPTION OF THE DRAWINGS

The above needs are at least partially met through provision of a waste disposal device and a subscription-based private civil security facilitation method incorporating the waste disposal device. These concepts are described in the following detailed description, particularly when studied in conjunction with the drawings, wherein:

FIG. 1 comprises a perspective view of an exemplary waste disposal system;

FIG. 2 comprises an exploded view of the waste disposal system of FIG. 1;

FIG. 3 comprises a cross-sectional view of the waste disposal system of FIG. 1;

FIG. 4 comprises a partial cross-sectional view of an alternative storage compartment used in the waste disposal system;

FIG. 5 comprises a cross-sectional view of an alternative waste disposal system;

FIG. 6 comprises a flow diagram as configured in accordance with various embodiments of the invention; and

FIG. 7 comprises a flow diagram as configured in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

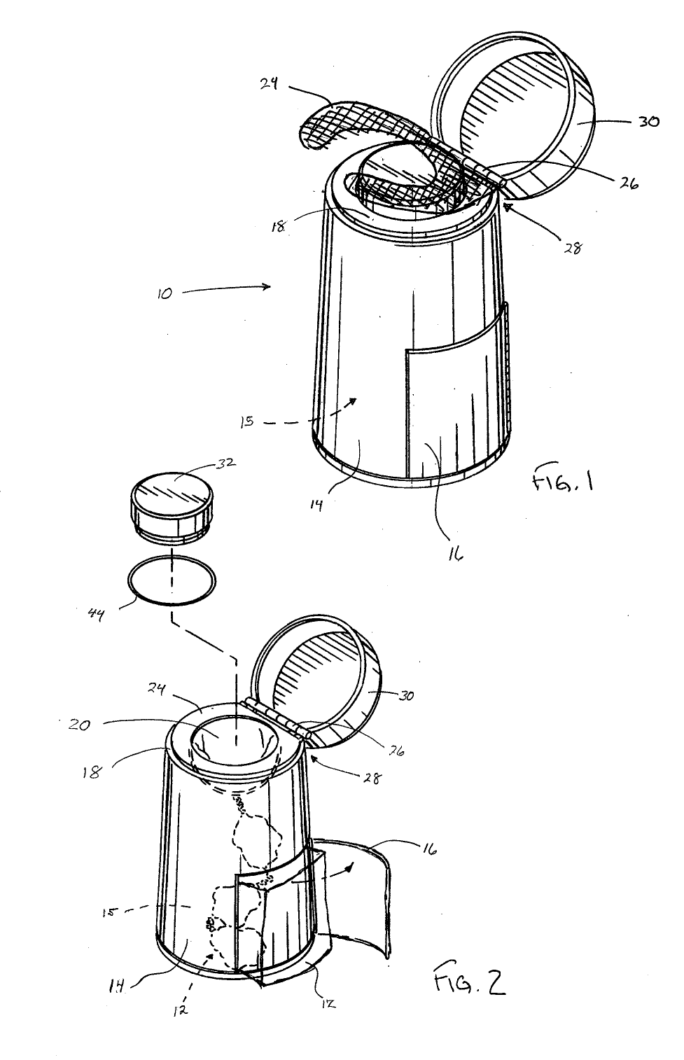

Referring to FIGS. 1-3, a first embodiment of a waste disposal device 10 is illustrated. The device 10 permits disposal of individual waste events (including both liquids and solids) in a generally self-contained system that is substantially free of liquid chemicals. The device 10 individually seals waste events in a storage compartment 12 to minimize heightened odors that may result from bulk accumulation of waste. The disposal device 10 is convenient for survival-related situations as well as various recreational uses (i.e., camping, homes, recreational vehicles, vehicles, cabins, shelters, and the like) because it is preferably portable and includes features that are commonly found on most household toilets. For purposes of this description, “portable” refers to a device that is of a size and weight that is movable (preferably when empty of waste) by a single adult human of average size. In this manner, the device 10 may be stored, such as on a shelf or in a closet, until needed and then replaced when its use is finished.

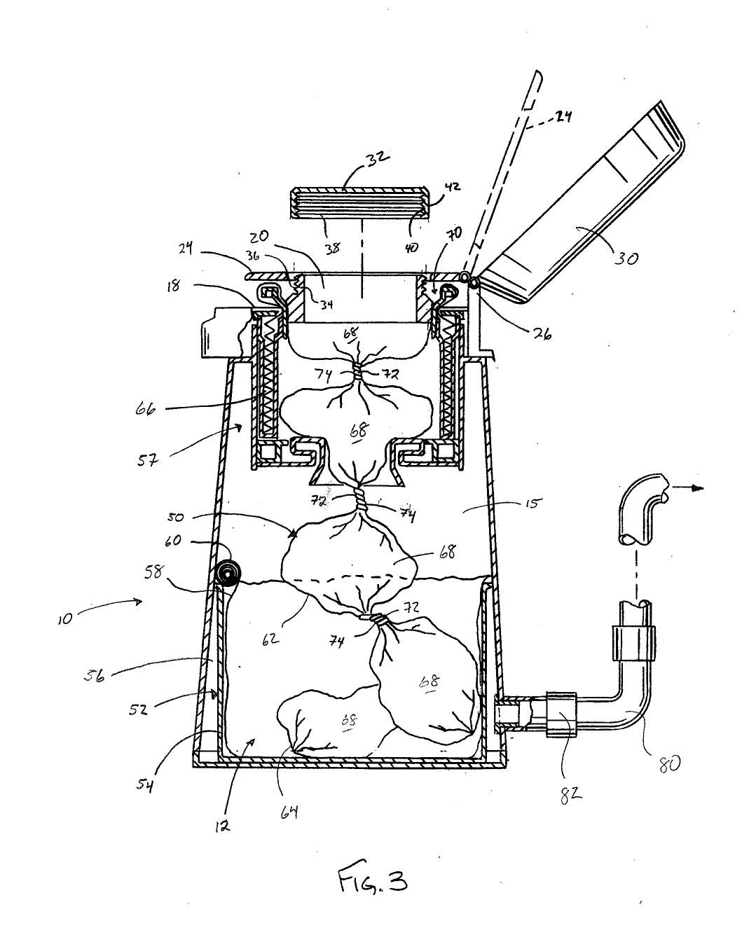

As generally shown in FIGS. 1 and 2, the device 10 includes a housing body 14 defining an internal space 15 sized to hold the storage compartment 12. The compartment 12 is preferably accessible through a hatch 16 so that the device 10 can be emptied when full. On an upper wall 18 of the housing 14 there is defined a through opening 20 leading into the storage compartment 12. The opening 20 preferably has a size and shape such that an individual may comfortably use the device 10 as a typical toilet. For example, the opening 20 may have a size ranging from about 8 to about 15 inches across.

Surrounding the opening 20 is a toilet seat 24 that is preferably connected to the housing 14 at a hinge joint 26 at a rear portion 28 of the housing 14. Other forms of attachment, or even a fully detachable toilet seat, may also be considered if desired. In one form, the toilet seat 24 may be generally horseshoe shaped as shown in FIG. 1, or it may be generally circular shaped as shown in FIG. 2. Other shapes of the seat 24 are also possible. Also connected to the housing 14, preferably through the hinge joint 26, is an upper lid 30 that is preferably sized and shaped to enclose both the seat 24 and the opening 20 when the device 10 is not in use. As with the toilet seat 24, other forms of attachment of the lid 30 are also possible. With the hinge joint 26, both the seat 24 and lid 30 may pivot up and down from the housing upper wall 18. These parts can be comprised of plastic though other materials may suffice as well. While the figures and discussion herein illustrate one form of the storage device 10, it should be appreciated by one skilled in the art that the device 10 is merely illustrative of an exemplary embodiment, and may vary in size, shape, operation, and/or configuration.

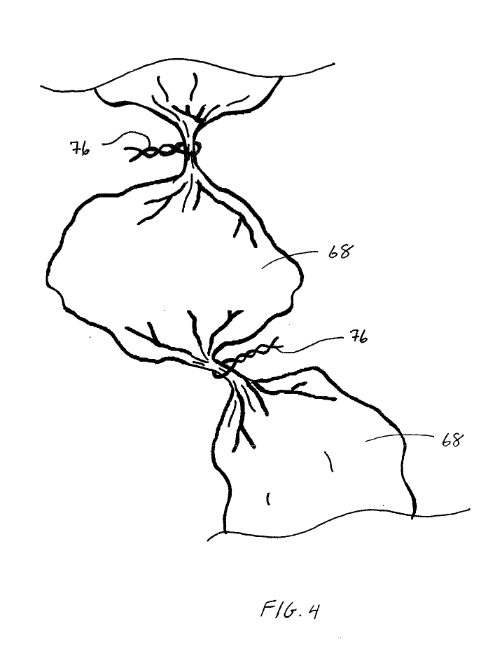

As shown in FIGS. 2 and 3, the housing 14 may also include an optional sealing cover 32 that seals the opening 20 when the device 10 is not in use to provide additional or enhanced protection from unwanted odors. In one form, the sealing cover 32 is threadably mated with the opening 20. For instance, as best shown in FIG. 3, surrounding the opening 20 is an upstanding lip 34 having male or external threads 36 thereon that threadably mate with female or internal threads 38 disposed on an internal surface 40 of a cover side wall 42. If desired, a seal member 44, such as an O-ring seal, may also be employed to help further seal the opening 20 when not in use. The threaded cover 32 and seal member 44 should generally provide a greater level of odor containment over typical hinge covers that are commonly used on household toilets, garbage pails, cans, or other waste containers.

Referring to FIG. 3, the storage compartment 12 preferably includes both a flexible portion 50 and a rigid portion 52. The flexible portion 50 has a variable size and configuration to individually seal waste events, and the rigid portion 52 has a predetermined size and configuration to retain a plurality of the individually sealed waste events contained within the flexible portion 50.

In one form, the rigid portion 52 includes a container 54, such as a basket, drawer, tank, drum, or other holding apparatus, that is housed within a lower portion 56 of the body 14. Preferably, the container 54 is accessible through the hatch 16 so that the storage compartment 12 may be removed when full.

Optionally, the container 54 may include one or more secondary containment systems 58 to help protect and secure waste events within the container 54. This added layer of protection in the storage compartment 12 generally provides better hygiene and also minimizes the risk of waste spills. In one form, the secondary containment system 58 may be one or more layers of a plastic or other flexible lining on an inside surface of the container 54. The plastic may be formed from extruded or laminated polyethylene, polypropylene, high density polyethylene, charcoal infused laminates, and/or other suitable plastics. If desired, the secondary containment system 58 may be pre-supplied in a replaceable module 60 so that it may be easily withdrawn from and fitted into an empty container 54. When the storage container is full and needs to be emptied, the container 54 may be accessed through the hatch 16 and the secondary containment system 58 may be removed along with the flexible portion 50 wrapped or secured therein. In this manner, the waste events are preferably double-bagged between the flexible portion 50 and the secondary containment system 58. This double layer of protection ensures easy, safe, and convenient disposal of the waste events.

Referring again to FIG. 3, the flexible portion 50 of the storage compartment 12 is a generally tubular flexible material 62 that is sealed at one end 64 and open at the other end in order to receive waste events. In one exemplary form, the flexible tubular material 62 is initially supplied in a replaceable cartridge 66 that may be inserted into an upper portion 57 of the housing body 14 in alignment with the opening 20. In use, a portion of the material 62 is withdrawn from the cartridge 66 so that the plastic's sealed end 64 is draped under the opening 20. In this configuration, the material 62 is positioned to receive the first waste event. Once received, the waste event is then isolated in individually sealed portions 68 of the material 62 by a sealing or isolating mechanism, such as an actuator 70. Once sealed or isolated, the individual portion 68 may then fall or be pushed into the container 54 through gravity or other forces. At the same time, a new portion of the material 62 is withdrawn from the cartridge 66 either from the waste event falling into the container 54 or other arrangement to prepare the device 10 for the next waste event.

In one embodiment, the individually sealed portions 68 of the storage container 12 are formed by manually rotating the actuator 70 such that the material 62 is twisted to form a twisted portion 72 that seals and defines a lower end of the sealed portion 68. Once a waste event is deposited in the material 62, the manual actuator 70 is again rotated so that the material 62 is again twisted to form a second twisted portion 74 to define an upper end of the sealed portion 68. As shown in the figures, the lower twisted portion 72 of one sealed portion 68 can also comprise the upper twisted portion 74 of a second sealed portion. In one form, the twisted portions 72 and 74 may each be formed through rotating the actuator 70 in the same direction (i.e., either clockwise or counter-clockwise). Alternatively, one twisted portion may be formed through a clockwise rotation of the actuator 70 and the other twisted portion may be formed through a counter-clockwise rotation of the actuator such that the sealing mechanism alternates directions between deposits.

The combination of the first and second twisted portions 72 and 74 preferably form the individually sealed portion 68, which is generally in the form of a pouch, wrap, or the like. (It will be appreciated that the initial sealed portion 68 will preferably only have a single twisted portion 74 on an upper end because the opposite end is the sealed end 64 of the material 62.) The waste event will be retained and sealed in the pouch formed between the spaced twisted portions 72 and 74. As shown, subsequent waste events will preferably be sealed in a similar fashion within formed individual portions 68 that are connected in a series along a length of the material 62. In this manner, each waste event is isolated in a separate portion 68 of the storage compartment 12. The connected series of waste events fall or are pushed into the container 54 for storage and later removal from the device 10.

Turning to FIG. 4, an alternative sealing mechanism is illustrated that employs fasteners 76 with the individually sealed portions 68. The fasteners 76 may be used in conjunction with the twisted portions 72 and 74, or as illustrated in FIG. 4, be used without twisting the material 62. If used together with the twisted portions, the fasteners 76 provide redundancy to the isolated portions 68 through additional or enhanced sealing. If used without twisting the material 62, the fasteners 76 hold the material 62 together in a tight gathering to form the individually sealed portions 68.

The fasteners 76 may include a wide variety of devices that clamp, seal, fasten, hold, squeeze, or otherwise form the ends of the individual portions 68. As shown, the fasteners 76 are a tie-type system, such as a metal or wire twist tie. However, the fasteners 76 may employ or include other types of holding devices, such as, but not limited to, spring clamps, bands, adhesive, peel seals, zippers, zip-locks, slider mechanisms, Velcro, screws, or other devices to gather and hold the material 62 in a tight fashion between the individual portions 68.

The flexible tubular material 62 may be any flexible plastic material that can be formed into a generally tubular structure and sealed at one end thereof. Preferably, the material 62 has sufficient strength to retain waste events and is generally non-permeable to liquids (i.e., water) and odors. Examples of the suitable plastic material include extruded and/or laminated plastics such as polyethylene, polypropylene, high density polyethylene, charcoal infused laminates, and the like. The plastic material 62 may also include a double bagged structure or comprise a laminate of multiple layers. In order to hide the contents of the storage container 12, the material 62 may be opaque or colored. While the above plastics are examples of material suitable for the flexible tubular material 62, other types of flexible or formable material may also be suitable.

Optionally, the device 10 may be vented to a remote location through a vent line 80 that provides an exhaust communication between the device internal space 15 and an external environment remote from the waste device. If vented, the device 10 preferably has a negative pressure within the internal space 15 such that odors do not escape the device 10 to an adjacent environment, but are instead vented through the vent line 80 away from the immediate vicinity. To this end, a vacuum pump or other suitable device (not shown) may be employed if needed along with the vent line 80 to form the negative pressure within the internal space. As the device 10 is preferred to be portable, the vent line 80 also preferably includes a removable connection 82 where the device 10 may be easily connected and disconnected to the vent line 80 as needed.

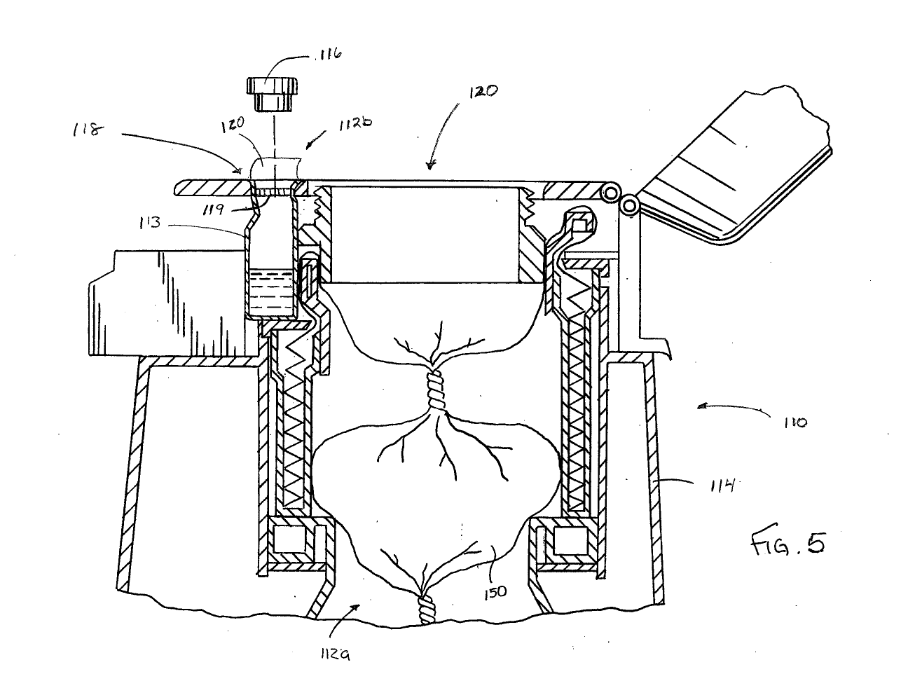

Referring to FIG. 5, an alternative waste disposal device 110 is illustrated. In this form, the device 110 is configured to separate liquid waste events and solid waste events into distinct storage containers 112a and 112b. In this manner, each type of waste event can be disposed of or used individually as needed.

Similar to the previous embodiment, the device 110 includes a housing body 114 with a through opening 120 that leads to the storage compartments 112a and/or 112b. The opening 120 preferably has a size and shape such that an individual may comfortably use the device 110 as a typical toilet and provide waste events in each of the appropriate storage containers 112a or 112b as needed. As with the previous embodiment, the general features described herein are merely illustrative of an exemplary embodiment, and may vary in size, shape, and/or configuration.

The first waste storage container 112a is preferably similar to the above described storage container 12 and includes a flexible portion 150 and a rigid portion (not shown). Therefore, the first container 112a functions in a manner similar to that previously described and will not be discussed further with this embodiment.

The second waste storage container 112b is preferably in the form of a sealable container 113 that is positioned on an upper end 118 of the housing body 114. The container 113 can be of any configuration, such as a more rigid construction (i.e., a bottle, jar, and the like) or a more flexible, variable construction (i.e., a plastic bag, zip-lock bag, pouch, sachet, or the like). As illustrated in FIG. 4, the container 113 is a jar that is sealable and/or re-sealable via a lid or cover 116. The storage container 112b shown herein is merely an exemplary illustration; it will be appreciated by one skilled in the art that the shape, size, volume, configuration, and placement of the container 112b on the housing body 114 may vary as needed to accommodate both male and female users and/or type of waste.

Optionally, the storage container 112b may include a strainer 119 to filter solid waste events from entering the container 113. The strainer 119 may be any suitable filter material, such as a mesh, screen, paper, woven, fabric, or like material. If desired, the storage container 112b may also include a guard 120 having a predetermined size and configuration to minimize splashing and to help direct liquid waste events into the storage container 112b.

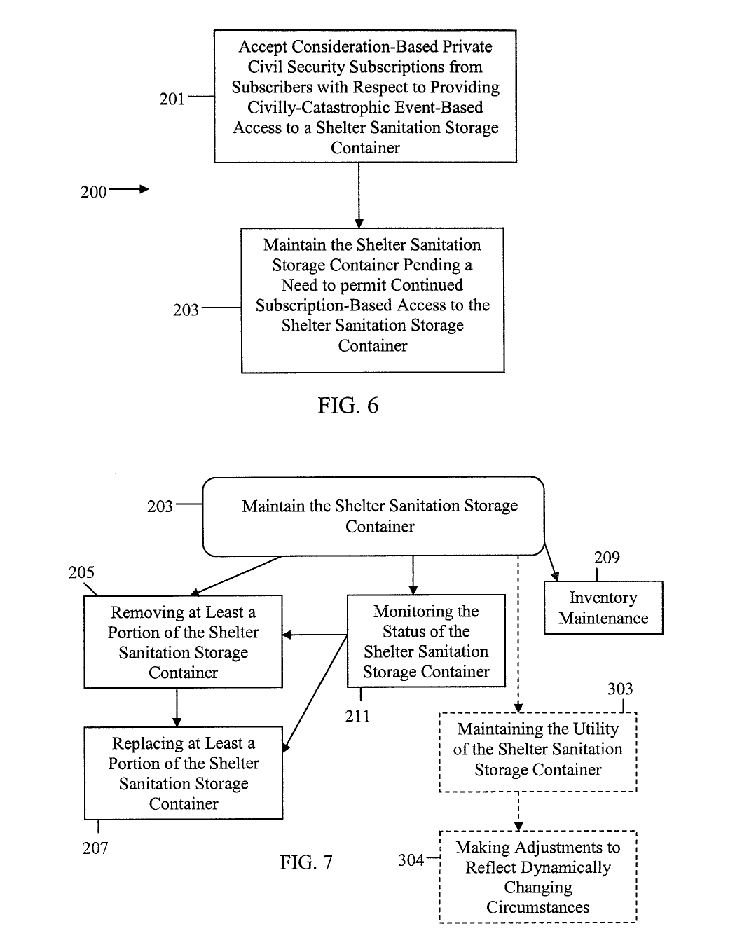

Turning to FIGS. 6 and 7, one exemplary use of the waste disposal devices 10 and 110 are illustrated as part of a survival-related application. For example, a consideration-based private civil security subscription is accepted from subscribers with respect to providing civilly-catastrophic event-based access for an authorized beneficiary to a shelter sanitation storage container, which may be the waste disposal devices 10 or 110. Then, the shelter sanitation storage container is maintained pending a need to permit continued subscription-based access to shelter sanitation storage container. In this manner, the subscriber and/or authorized beneficiary is provided with access to a sanitary disposal system if society's infrastructure that provides such services becomes disrupted or if such facilities are needed in a shelter context where ordinary sanitary disposal systems are not otherwise available or operable.

These steps are facilitated without dependency upon governmental oversight, participation, or control. The specifics of the shelter sanitation storage container or the level of maintenance provided can vary with the needs and requirements of the subscribers or authorized beneficiaries. Therefore, an individual can take important steps to bring a considerably improved measure of security into their lives that sanitation needs will be provided for without having to effectively become a full-time survivalist. Such individuals can, in short, continue to enjoy their chosen vocations and standard of living knowing that they will have extraordinary access to at least a sanitation system to dispose of waste events should a civilly-catastrophic event indeed be visited upon them.

Referring again to the figures, and in particular to FIG. 6, these teachings provide generally a system or process 200 for accepting 201 consideration-based private civil security subscriptions from subscribers with respect to providing civilly-catastrophic event-based access for an authorized beneficiary to at least a shelter sanitation storage container, which as mentioned above, may be the waste disposal devices 10 or 110.

These teachings provide for a subscription-based approach. As used herein, the term “subscription” shall be understood to refer to and encompass a variety of legal mechanisms. Relevant examples include, but are not limited to, the subscription mechanisms that are described in U.S. patent application Ser. No. 11/384,037, which is incorporated herein in its entirety.

These teachings also readily encompass the notion of a given subscriber providing such a subscription for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist.

As noted, these subscriptions relate to providing access to the shelter sanitation storage container in the event of a civilly-catastrophic event. Such access may be predicated, if desired, upon a requirement that the civilly-catastrophic event be one that persists in substantial form for more than a predetermined period of time (such as one hour, one day, one week, and so forth) or that causes at least a predetermined amount or degree of infrastructure impairment or other measurable impact of choice.

As used herein, “civilly-catastrophic event” will be understood to refer to an event that substantially and materially disrupts a society's local, regional, and/or national infrastructure and ability to provide in ordinary course for the at least one life-sustaining resource that is the subject of the subscription. Such a civilly-catastrophic event can include both a precipitating event (which may occur over a relatively compressed period of time or which may draw out over an extended period of time) as well as the resultant aftermath of consequences wherein the precipitating event and/or the resultant aftermath include both the cause of the infrastructure interruption as well as the continuation of that interruption.

A civilly-catastrophic event can be occasioned by any of a wide variety of natural and/or non-naturally-caused disasters. Examples of natural disasters that are potentially capable of initiating a civilly-catastrophic event include, but are not limited to, extreme weather-related events (such as hurricanes, tsunamis, extreme droughts, widespread or unfortunately-targeted tornadoes, extreme hail or rain, and the like, flooding, and so forth), extreme geological events (such as earthquakes, volcanic activity, and so forth), extreme space-based events (such as collisions with comets, large asteroids, and so forth as well as extreme solar flares and the like), extreme environmental events (such as widespread uncontrolled fire or the like), and global or regional pandemics, to note but a few.

Examples of non-naturally-caused disasters capable of initiating a civilly-catastrophic event include both unintended events as well as intentional acts of aggression such as war, terrorism, madness or the like. Examples of non-naturally-caused disasters capable of such potential scale include, but are not limited to, nuclear-related events (including uncontrolled fission or fusion releases, radiation exposure, and so forth), acts of war, the release of deadly or otherwise disruptive biological or chemical agents or creations, and so forth.

Again referring to FIG. 6, this process 200 then provides for maintaining 203 the shelter sanitation storage container pending a need to permit continued subscription-based access to the shelter sanitation storage container, which may be in response to the occurrence of a catastrophic event. This, of course, can comprise maintaining the resource on behalf of the subscriber and/or on behalf of another authorized beneficiary.

In one example, the need may include the shelter sanitation storage container achieving at least a predetermined capacity of waste events, such as the waste device 10 or 110 reaching a predetermined capacity (i.e., half-full, three-quarter full, full, or other predetermined level) in the storage compartments 12, 112a, or 112b. Of course, the specifics of the need or maintenance will vary with respect to the nature of the shelter sanitation storage container being maintained, the subscriber, and/or the authorized beneficiary.

As one example, and referring to FIG. 7, maintaining 203 the resource can comprise a variety of actions. For example, maintaining may include 205 removing at least a portion of the shelter sanitation storage container, such as removing the entire storage compartment 12, 112a, or 112b, removing just the flexible portion 62, and/or removing the secondary containment system 58 along with the flexible portion 62. Maintaining may also involve 207 replacing at least a portion of the shelter sanitation storage container, such as replacing the flexible portion cartridge 66, the flexible portion 62, the rigid portion 54, and/or the module 60 for the secondary containment system 58. Maintaining may also involve 209 the re-supply of an inventory of shelter sanitation storage containers or any portion thereof. For instance, maintenance may include ensuring and providing the subscribers and/or authorized beneficiaries with a sufficient supply of storage compartments 12, 112a, or 112b (or other supplies for such devices) so that unused storage compartments or supplies are readily available when needed.

The maintenance may be provided upon notification from the subscribers and/or authorized beneficiaries. To this end, maintenance will be provided upon subscriber or authorized beneficiary providing notice that the above described need is satisfied. This notice may be a phone call, a wireless signal, an internet signal, or the like. Alternatively, maintenance may also be provided automatically at set periodic intervals (i.e., weekly, monthly, etc.) or via monitoring 211 of the status of one or more shelter sanitation storage containers. To this end, the shelter sanitation storage container may include sensors, such as level or weight sensors, to indicate when the need has been met. For instance, the sensor would indicate that the storage container 12, 112a, or 112b has reached the predetermined capacity. Such sensors would communicate via phone lines, wireless communication, Internet, or other suitable communication methods to indicate that the above described need has been met.

Such maintenance 203 can also optionally comprise maintaining the utility 303 of the shelter sanitation storage container or making adjustments 304 to the shelter sanitation storage container to reflect dynamically changing circumstances that may occur during the consideration-based private civil security subscription period. As one illustration, a new product or new supplies may become available that is particularly useful in providing, storing, and disposing of waste events or other sanitation. In such a case, maintaining 203 can readily accommodate updating the shelter sanitation storage container to include this new product or new supplies. Accordingly, this step of making adjustments 304 can readily comprise one or more of removing a particular shelter sanitation storage container or a portion thereof (as when a better substitute becomes available, when the container or portion thereof is shown to be less effective for its intended purpose than was originally thought, and so forth), adding additional shelter sanitation storage containers (as when it becomes subsequently understood that more containers are desirable to achieve suitable sanitary disposal of waste events), adding at least one new stored product that is not already stored (as illustrated in the example provided above), and so forth.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

What is claimed is:

1. A waste storage device comprising:
a storage compartment;
a toilet seat having an opening providing access to the storage compartment; and
a sealing mechanism operative to isolate deposited waste events within individually sealed portions of the storage compartment, such that the storage compartment will retain a plurality of individually sealed portions in a connected series along a length of the storage compartment.

2. The waste storage device of claim 1, wherein the storage compartment comprises a flexible material.

3. The waste storage device of claim 2, wherein the flexible material is a plastic.

4. The waste storage device of claim 2, wherein the individually sealed portions of the storage compartment comprise a pouch formed from the flexible material between spaced twists of the storage compartment, the pouch being formed by the sealing mechanism.

5. The waste storage device of claim 2, further comprising a cartridge containing the flexible material and configured for removal of the flexible material therefrom during use.

6. The waste storage device of claim 1, further comprising a cover operative to seal the opening when the waste storage device is not in use in order to substantially isolate an interior of the waste storage device from the surroundings.

7. The waste storage device of claim 6, wherein the cover is rotatably threaded to the waste storage device.

8. The waste storage device of claim 7, further comprising a seal received between the cover and the waste storage device.

9. The waste storage device of claim 1, further comprising a size and weight such that the waste storage device is movable by a single adult human of average size.

10. The waste storage device of claim 1, wherein the waste storage device includes a vent providing an exhaust communication between an interior of the waste storage device and an environment remote from the waste storage device.

11. The waste storage device of claim 10, wherein the interior has a negative pressure.

12. The waste storage device of claim 1, wherein the sealing mechanism comprises a manual actuator to form the individually sealed portions of the storage compartment.

13. The waste storage device of claim 1, wherein the storage compartment is substantially free of liquid chemicals.

14. The waste storage device of claim 1, further comprising a second storage compartment positioned to receive liquid waste events, and wherein the storage compartment is positioned to receive solid waste events.

15. The waste storage device of claim 1, wherein the storage compartment includes a plurality of plastic layers.

16. A method comprising:
accepting consideration-based private civil security subscriptions from subscribers with respect to providing civilly-catastrophic event-based access to a shelter sanitation storage container; and
maintaining the shelter sanitation storage container pending a need to permit continued subscription-based access to the shelter sanitation storage container.

17. The method of claim 16, wherein the need comprises a shelter sanitation storage container achieving at least a predetermined capacity.

18. The method of claim 17, wherein the maintaining comprises removing at least a portion of the shelter sanitation storage container.

19. The method of claim 18, wherein the maintaining further comprises replacing at least a portion of the shelter sanitation storage container.

20. The method of claim 17, wherein maintaining includes monitoring a status of the shelter sanitation storage container.

21. The method of claim 16, wherein the shelter sanitation storage container comprises a waste storage container movable by a single adult human of average size.

22. The method of claim 21, wherein the waste storage container movable by a single adult human of average size includes:
a storage compartment;
a toilet seat having an opening providing access to the storage compartment; and
a sealing mechanism operative to isolate deposited waste events within individually sealed portions of the storage compartment, such that the storage compartment will retain a plurality of the individually sealed portions in a connected series along a length of the storage compartment.

23. The method of claim 22, wherein the maintaining comprises removing the storage compartment from the waste storage container upon achieving at least a predetermined level of waste in the storage compartment.

24. The method of claim 23, wherein the maintaining further comprises replacing the storage compartment having the predetermined level of waste with another storage compartment.
